(19) United States
(12) Patent Application Publication
(10) Pub. No.: US 2026/0265187 A1
(43) Pub. Date: Sep. 10, 2026

(54) METHOD FOR PREPARING ARYLVINYLSULPHONES

(71) Applicant: KEMIRA OYJ, Helsinki (FI)

(72) Inventor: Yogesh Laxman BOROLE, Bharuch District (IN)

(21) Appl. No.: 19/166,365

(22) PCT Filed: Mar. 7, 2024
(86) PCT No.: PCT/EP2024/056047

§ 371 Date: Sep. 17, 2025

(30) Foreign Application Priority Data
Mar. 24, 2023 (IN) 202331021008

(51) Int. Cl. C07C 315/04

(52) U.S. Cl. CPC C07C 315/04

(57) ABSTRACT

A method for preparing arylvinylsulphones comprises reacting an aryl sulphonyl halide with a reactive solvent selected from (meth)acrylonitrile or alkyl (meth)acrylate in the presence of a Cu(I) catalyst in a first reaction mixture to form an intermediate product, wherein the Cu(I) catalyst is uncomplexed. Unreacted reactive solvent is separated from the first reaction mixture to form a second mixture. A low polarity solvent is added to the second mixture to precipitate the Cu(I) catalyst and dissolve the intermediate product to form a third reaction mixture. A base is added to the third reaction mixture, wherein the intermediate product undergoes a base-catalysed elimination of the halogen atom from the intermediate product to form an arylvinylsulphone compound preferably under cooling. The arylvinylsulphone compound, is separated from the third reaction mixture.

### TECHNICAL FIELD

The present invention relates to a method for preparing arylvinylsulphones from aryl sulphonyl halides.

### BACKGROUND

Arylvinylsulphones have various industrial uses. For example, 3-(aryl sulphonyl)-propene nitriles may be used as biocides in industrial processes as disclosed in WO 2019/042984 A1 and WO 2019/042985 A1.

A number of methods exist for preparing arylvinylsulphones. Many of these methods employ solvents that are environmentally harmful, volatile, flammable and/or scarcely available, such as acetonitrile. Furthermore, the existing processes comprise a number of steps which increase the complexity of the processes. There are also only limited possibilities for recycling any of the chemicals used in existing processes.

An early example of such a process is described by Asscher and Vofsi in J. Chem. Soc. 1964, 4962-4971. This describes the addition of sulphonyl chlorides to vinylic monomers and other olefins. Exemplified are reactions of sulphonyl chlorides with olefins using cupric chloride-based catalyst complexes with triethylammonium chloride. Following an addition reaction with the olefin there is a base catalysed dehydrohalogenation.

WO 2020/094917 A1 describes a process for the preparation of aryl sulphonyl propene nitriles. This process also proceeds by an addition reaction in which a sulphonyl halide is reacted with acrylonitrile and a catalyst, followed by a base catalysed elimination so as to form the desired product. According to this disclosure a catalyst complex is prepared immediately before addition to the reaction mixture by the complexation of an inorganic halide with a salt of an organic compound.

Exemplified is the preparation of a cuprous iodide-based catalyst complex with triethylamine hydrochloride in acetonitrile. Tosyl chloride is then reacted with acrylonitrile in the presence of this catalyst complex.

These processes suffer from some of the above disadvantages. It is therefore an aim of the present invention to provide an improved method for preparing arylvinylsulphones which minimizes or overcomes at least some of the disadvantages that arise in the existing processes.

### SUMMARY OF THE INVENTION

In a first aspect the present invention provides a method for preparing a compound of Formula (I)

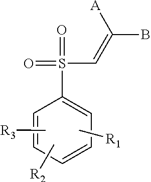

wherein
R1, R2, and R3 independently represent a hydrogen atom; a halogen atom; a hydroxy group; an alkyl group; a hydroxy alkyl group; a haloalkyl group; an alkoxy group having 1 to 4 carbon atoms; an acyl group: an amino group; an alkylamino group or an acylamido group having 1 to 10 carbon atoms;
A represents a hydrogen atom; a C1-C5 alkyl group; or an alkoxycarbonyl group; and
B represents a nitrile group, a carboxylic acid group, a carboxylic acid ester group or a carboxylic acid amide group.

The method comprises the steps of:
(a) reacting an aryl sulphonyl halide with a reactive solvent selected from (meth)acrylonitrile or alkyl (meth)acrylate in the presence of a Cu(I) catalyst in a first reaction mixture to form an intermediate product, wherein the Cu(I) catalyst is uncomplexed;
(b) separating unreacted reactive solvent from the first reaction mixture to form a second mixture;
(c) adding a low polarity solvent to the second mixture to precipitate the Cu(I) catalyst and dissolve the intermediate product to form a third reaction mixture;
(d) adding a base to the third reaction mixture, wherein the intermediate product undergoes a base-catalysed elimination of the halogen atom from the intermediate product to form the compound according to Formula (I), preferably under cooling; and
(e) separating the compound according to Formula (I) from the third reaction mixture.

It has surprisingly been found that using an uncomplexed Cu(I) catalyst improves the method for preparing the compound of Formula (I). Higher yields are obtainable. It is possible to operate the method at a lower temperature. The reactant compositions are simplified avoiding the need for any pre-treatment steps to produce a catalyst complex, which was hitherto thought to be essential in the method. The simplification of the method also allows reactant and catalyst materials to be readily recycled.

In the context of the present invention, an uncomplexed Cu(I) catalyst comprises a monovalent copper ion, optionally in the presence of a counterion, to which is attached no ligands. The Cu(I) catalyst may be a Cu(I) halide catalyst, for example with an iodide, bromide or chloride counterion. A preferred catalyst is a Cu(I) chloride catalyst. The Cu(I) halide is typically added to the first reaction mixture as a solid.

In one embodiment of the invention, R1 in Formula (I) represents a hydrogen atom, an alkyl group comprising 1 to 4 carbon atoms or an alkoxy group having 1 to 4 carbon atoms. R1 may also represent an acyl group comprising 1 to 4 carbon atoms. R1 can be, for example, a methyl group, an ethyl group, a propyl group; a butyl group, a methoxy group, an ethoxy group, a propoxy group, an isopropoxy group, a n-butoxy group, or a tertiary butoxy group. Preferably, R1 represents a methyl group in the 4-position.

R2 and R3 in Formula (I) represent independently from each other and independently from R1 a hydrogen atom, an alkyl group comprising 1 to 4 carbon atoms or an alkoxy group having 1 to 4 carbon atoms. R2 and/or R3 may be, for example, a methyl group, an ethyl group, a propyl group, a butyl group, a methoxy group, an ethoxy group, a propoxy group, an isopropoxy group, a n-butoxy group, or a tertiary butoxy group. Preferably, both R2 and R3 represent a hydrogen atom.

According to one embodiment R1, R2 and/or R3 in Formula (I) may represent independently from each other a halogen atom, such as chlorine, fluoride or bromide; a hydroxy group; a hydroxy alkyl group preferably comprising 1-4 carbon atoms; a haloalkyl group preferably comprising 1-4 carbon atoms and/or fluoro-substituents, such as trifluoromethyl; an amino group; an alkylamino group having 1 to 10 carbon atoms; or an acylamido group having 1 to 10 carbon atoms.

In a preferable embodiment R1 represents a methyl group in the 4-position of the aryl, and R2 and R3 both represent hydrogen.

According to one embodiment, group A in Formula (I) represents a C1-C5 alkyl group or a hydrogen atom. Group A may be, for example, a methyl group, an ethyl group, a propyl group; a butyl group, or a pentyl group. Preferably, the group A is a hydrogen atom.

According to one embodiment, group B in Formula (I) represents a nitrile group; a C1-C5 carboxylic acid group, a C1-C5 carboxylic acid ester group or a C1-C5 carboxylic acid amide group.

According to one embodiment of the invention the compound of Formula (I) may be an arylsulphonylpropene nitrile. The compound of Formula (I) may be selected from a group consisting of the compound according to Formula (I) is 3-[(4-methylphenyl) sulphonyl]-2-propenenitrile, 3-phenylsulphonyl-2-propenenitrile, 3-[(4-fluorophenyl) sulphonyl]-2-propenenitrile, 3-[(2,4-dimethylphenyl) sulphonyl]-2-propenenitrile, 3-[(4-trifluormethylphenyl)-sulphonyl]-2-propenenitrile, 3-[(3,4-dimethylphenyl) sulphonyl]-2-propenenitrile, 3-(2,5-dimethylphenyl) sulphonyl-2-propenenitrile, 3-[(2,4,6-trimethylphenyl) sulphonyl]-2-propenenitrile, 3-(4-methoxyphenyl) sulphonyl-2-propenenitrile, methyl (3-[(4-methyl-phenyl) sulphonyl]prop-2-enate, 3-(4-acetylphenyl) sulphonyl-2-propenenitrile, and any of their isomers. According to one preferable embodiment of the present invention the compound according to Formula (I) is selected from a group consisting of 3-[(4-methylphenyl) sulphonyl]-2-propenenitrile; 3-phenylsulphonyl-2-propenenitrile; 3-[(4-trifluormethylphenyl)-sulphonyl]-2-propenenitrile; 3-[(2,4,6-trimethylphenyl) sulphonyl]-2-propenenitrile; 3-(4-methoxyphenyl) sulphonyl-2-propenenitrile; 3-[(4-methylphenyl) sulphonyl]prop-2-enate; and any of their isomers. Preferably the compound of Formula (I) may be 3-[(4-methylphenyl) sulphonyl]-2-propenenitrile.

In the method of the present invention a first reaction mixture is formed in solution form by mixing the components of the first reaction mixture with each other. The first reaction mixture may be formed at a temperature of 15-40° C., preferably 20-30° C., more preferably 25-30° C. It is advantageous that the first reaction mixture may be formed at room temperature and no heating is necessary.

The first reaction mixture comprises the aryl sulphonyl halide, the reactive solvent and the Cu(I) catalyst.

The reactive solvent for the first reaction mixture is selected from (meth)acrylonitrile or alkyl (meth)acrylates, such as methyl acrylate or methyl methacrylate. Preferably the reactive solvent is acrylonitrile. According to one preferable embodiment of the present invention the first reaction mixture is free of all other solvents than the reactive solvent. According to this embodiment, no other solvents than the reactive solvent are added to the first reaction mixture.

The Cu(I) catalyst, as defined above, is dissolved in the reactive solvent, whereby the first reaction mixture in solution form is obtained. The reactive solvent functions both as a reactant and a solvent in the method according to the invention. The reactive solvent is preferably added in excess, which ensures the dissolution of the other components of the first reaction mixture. It has been found that the reactive solvent is able to dissolve the Cu(I) catalyst effectively, even at room temperature. As noted above, no heating for forming the first reaction mixture is thus required. Furthermore, an excess of reactive solvent facilitates the desired reaction for obtaining the intermediate product for compound of Formula (I). This simplifies the process and improves the conversion and/or yield of the process. However, only a small excess of reactive solvent may be added or needed.

The aryl sulphonyl halide may be added to the first reaction mixture as one single dose, or as a plurality of successive doses, such as two, three or more successive doses. A continuous addition may also be possible. Preferably aryl sulphonyl halide is added to the first reaction mixture as a single dose. The aryl sulphonyl halide may be benzene sulphonyl halide, alkyl substituted benzene sulphonyl halide or halo-substituted benzene sulphonyl halide, such as toluene sulphonyl halide, xylene sulphonyl halide, 4-methoxybenzene sulphonyl halide or 4-chlorobenzene sulphonyl halide, preferably toluene sulphonyl chloride or toluene sulphonyl bromide, more preferably toluene sulphonyl chloride. For example, the aryl sulphonyl halide may be 4-toluene sulphonyl halide, preferably 4-toluene sulphonyl chloride or 4-toluene sulphonyl bromide, more preferably 4-toluene sulphonyl chloride. Aryl sulphonyl halide may be added in amount (in mol) of 0.3-0.7 equivalents, preferably 0.4-0.6 equivalents, more preferably around 0.5 equivalents, relative to the amount (in mol) of the reactive solvent in the first reaction mixture. Aryl sulphonyl halide is dissolved in the first reaction mixture, under an endothermic process.

The amount of Cu(I) catalyst in the first reaction mixture may be 5-30 mol-%, preferably 10-25 mol-%, more preferably 15-20 mol-%, calculated from the amount of the aryl sulphonyl halide in the first reaction mixture.

After the addition of the aryl sulphonyl halide to the first reaction mixture, and its dissolution, the reaction is allowed to proceed at an elevated temperature, whereby an intermediate product is obtained. The reaction is a radical addition reaction. The reaction is preferably allowed to proceed at the elevated temperature, which is higher than the boiling point of the reactive solvent but below the boiling point of the reaction mixture. The reaction in the first reaction mixture may be allowed to proceed at the elevated temperature of 80-95° C., preferably 85-92° C., more preferably 85-87° C. The reaction is allowed to proceed until desired conversion of the intermediate product is obtained. The reaction time may be, for example 10-30 hours, preferably 10-24 hours, more preferably 12-16 hours.

After the reaction has proceeded to the desired conversion, the unreacted reactive solvent may be separated from the first reaction mixture to form the second reaction mixture. The reactive solvent may be separated by any suitable separation method, e.g. by distillation. According to one preferable embodiment, the reactive solvent may be separated from the first reaction mixture and then recycled back in the process to the step (a) where the first reaction mixture is formed. In this manner it is possible to reuse the unreacted reactive solvent in the preparation of the first reaction mixture and effectively reduce the amount of chemical waste which is produced. Advantageously, little or no purification of the separated reactive solvent is necessary.

After the separation of the unreacted reactive solvent a second reaction mixture is formed. This comprises the intermediate product and the Cu(I) catalyst. A low polarity solvent is then added to the second reaction mixture. This precipitates the Cu(I) catalyst from the second reaction mixture and dissolves the intermediate product, thereby forming a third reaction mixture. This step may be performed while the second reaction mixture is still at elevated temperature following the reaction in the first reaction mixture. Once the low polarity solvent has been added, the resulting third reaction mixture may be allowed to cool. At this stage, the precipitated Cu(I) catalyst may be removed from the third reaction mixture before further reactions take place.

The Cu(I) catalyst precipitate may be easily separated from the liquid phase of the third reaction mixture that comprises the low polarity solvent and the intermediate product, e.g. by filtration. Removal is preferably completed prior to step (d) so that the Cu(I) catalyst is not contaminated or oxidized. Removal at this stage allows the Cu(I) catalyst to be easily recycled back to step (a). Advantageously, purification or reduction of the catalyst is not necessary.

The low polarity solvent has a relative polarity of <0.4, preferably <0.3, or even <0.25, to form the third reaction mixture. For purposes of the present context, the relative polarity values given, for example, in Reichardt, C. (ed.) and Welton, T. (ed.), “Solvents and Solvent Effects in Organic Chemistry”, 4th Ed., 2011, Wiley-VCH Verlag Gmbh & Co., Weinheim, Appendix A, or in other similar handbooks, can be used. The low polarity of the low polarity solvent reduces its ability to mix with water, which simplifies the separation of the intermediate product from the aqueous phase. The low polarity solvent may be considered hydrophobic. The low polarity solvent may be free of carboxyl or hydroxyl groups. Low polarity solvents suitable for use in the present invention may be selected, for example, from a group consisting of ethyl acetate, butyl acetate, tetrahydrofuran, dioxane and toluene. According to one preferable embodiment the low polarity solvent is ethyl acetate.

After dissolving the intermediate product in the low polarity solvent for forming the third reaction mixture, and optional separation of Cu(I) precipitate, a base may be added to the third reaction mixture for elimination of the halogen atom from the intermediate product to form the compound according to Formula (I). The intermediate product undergoes an elimination reaction in the presence of the base, where the halogen atom is removed from the intermediate product, i.e. a base-catalysed elimination of the halogen atom, and the compound according to Formula (I) is obtained. The base used may be an inorganic base, an organic base or a combination of an inorganic and organic base, preferably an organic base. The elimination reaction is exothermic, and therefore the base is preferably added, and the elimination reaction is allowed to proceed under cooling. According to one preferable embodiment the third reaction mixture is maintained at a temperature of 15-40° C., preferably 20-35° C., more preferably 20-25° C., during the elimination of halogen atom from the intermediate product.

The base may be added to the third reaction mixture in an amount (in mol) of at least 1 equivalent, preferably at least 1.3 equivalent, relative to the amount of the dissolved intermediate in the second reaction mixture.

The base added to the third reaction mixture may comprise an inorganic base, preferably selected from a group comprising bicarbonates and carbonates of alkali metals and carbonates of alkaline earth metals or any mixtures thereof. For example, the inorganic base may be selected from sodium bicarbonate, sodium carbonate, potassium bicarbonate, potassium carbonate, lithium bicarbonate, lithium carbonate, caesium carbonate, calcium carbonate, magnesium carbonate or any mixture thereof. According to one preferable embodiment sodium bicarbonate or sodium carbonate is used as inorganic base.

Preferably, the base added to the third reaction mixture comprises an organic base, preferably selected from trialkylamines, such as triethylamine, trimethylamine; N-methylmorpholine; N-methylpyrrolidine; N,N-diisopropylethylamine (Hünig's base); 1,4-diazabicyclo[2.2.2]octane (DABCO); 1,8-diazabicyclo[5.4.0]undec-7-ene (DBU); or 1,5-diazabicyclo[4.3.0]non-5-ene (DBN), or any mixtures thereof. Triethylamine is preferred.

The base may also comprise a combination of an inorganic base and an organic base, as defined above.

According to one embodiment of the present invention, the third reaction mixture additionally comprises water. Water may be introduced into the third reaction mixture before addition of base. The elimination reaction proceeds when the base is added to the third reaction mixture.

In an alternative embodiment of the present invention, water is added to the third reaction mixture together with or after the addition of base. According to this embodiment the base is preferably a combination of an inorganic base and an organic base, as defined above. The base may comprise 0.8-0.95 equivalent, preferably 0.9-0.95 equivalent of an inorganic base and 0.05-0.2 equivalent, preferably 0.05-0.1 equivalent of an organic base, given as molar equivalents. The inorganic and organic base may be added to the third reaction mixture, or separately but simultaneously, or separately and successively. Preferably the inorganic base is added first, followed by addition of organic base.

In this alternative embodiment, the amount of water in the third reaction mixture may be 15-200 weight-%, preferably 40-150 weight-%, more preferably 55-100 weight-%, calculated from the amount of the base in the third reaction mixture. Efficient stirring of the third reaction mixture during the elimination reaction prevents the phase separation between the water and the low polarity solvent. The water content of the third reaction mixture enables a phase transfer process involving the organic base and inorganic base, if both present, that improves the efficiency of the base-catalysed elimination reaction.

After the elimination reaction is complete, the compound according to Formula (I) is separated from the third reaction mixture. The mixture contains immiscible aqueous and organic phases which allow the unwanted aqueous phase to be readily separated. The organic phase contains a precipitate of the compound according to Formula (I) which may be easily separated from the organic solvent phase, e.g. by filtration. The filtrate may be washed using an acid such as dilute HCl. A relatively pure compound according to Formula (I) may be obtained as a solid or semi-solid product. The product may be recrystallized from an alcohol such as a C1-C3 alcohol, preferably isopropyl alcohol. This is followed by filtration and evaporation of solvent. Recrystallisation typically recovers the E-isomer at a purity of at least 99.5%.

The term “comprises” as used throughout the description and claims herein means “includes or consists of”. The term denotes the inclusion of at least the features following the term and does not exclude the inclusion of other features which have not been explicitly mentioned. The term may also denote an entity which consists only of the features following the term.

### DETAILED DESCRIPTION OF THE INVENTION

The present invention will now be described in further detail, by way of example only with reference to the following Example.

### Example

All chemicals and reagents used in this example were reagent grade purity unless indicated otherwise.

### Preparation of Intermediate Product

At room temperature 2.74 g, Cu(I) chloride, 34 g p-toluene sulphonyl chloride and 20 g acrylonitrile were mixed together in a reactor. The mixture was heated to a temperature in the range 85 to 87° C. This temperature was maintained for 16 hours while stirring the mixture. Excess acrylonitrile was removed by vacuum distillation at a temperature in the range 72 to 92° C. 50 g ethyl acetate was added to the remaining mixture, which was then stirred and allowed to cool. Greenish-grey-white catalyst particles were thereby precipitated. The remaining mixture contained the intermediate product.

### Catalyst Removal

The catalyst precipitate was removed once the mixture reached approximately 45° C. This may be recycled after two washes in 5 g ethyl acetate followed by drying and storage under nitrogen.

### Elimination Reaction

After catalyst removal the reaction mixture was cooled to room temperature. 25 g water was added, followed by 18.2 g triethylamine. The temperature was maintained below 25° C. Two solvent layers formed. The aqueous layer was removed. The organic layer contained a yellowish white precipitate of p-toluene sulphonyl propenenitrile product. This was separated by filtration and washed twice with dilute HCl.

### Recrystallisation

The product following the washing steps contained residual solvent which was allowed to evaporate. 30 g isopropyl alcohol was added to the solid product, which was stirred well to ensure full dissolution. The solution was left at refrigeration temperature of 6° C. overnight to allow the formation of crystals. The crystals were filtered and washed with chilled isopropyl alcohol followed by drying at 45° C. under nitrogen.

A yield of 73% was obtained.

Analysis of the product showed E-3-[(4-methylphenyl) sulphonyl]-2-propenenitrile.

1. A method for preparing a compound of Formula (I): 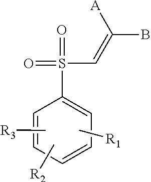
wherein
R1, R2, and R3 independently represent a hydrogen atom; a halogen atom; a hydroxy group; an alkyl group; a hydroxy alkyl group; a haloalkyl group; an alkoxy group having 1 to 4 carbon atoms; an acyl group; an amino group; an alkylamino group or an acylamido group having 1 to 10 carbon atoms;
A represents a hydrogen atom; a C1-C5 alkyl group; or an alkoxycarbonyl group;
B represents a nitrile group, a carboxylic acid group, a carboxylic acid ester group or a carboxylic acid amide group;
the method comprising steps of:
(a) reacting an aryl sulphonyl halide with a reactive solvent selected from (meth)acrylonitrile or alkyl (meth)acrylate in the presence of a Cu(I) catalyst in a first reaction mixture to form an intermediate product, wherein the Cu(I) catalyst is uncomplexed;
(b) separating unreacted reactive solvent from the first reaction mixture to form a second mixture;
(c) adding a low polarity solvent to the second mixture to precipitate the Cu(I) catalyst and dissolve the intermediate product to form a third reaction mixture;
(d) adding a base to the third reaction mixture, wherein the intermediate product undergoes a base-catalysed elimination of the halogen atom from the intermediate product to form the compound according to Formula (I), preferably under cooling; and
(e) separating the compound according to Formula (I) from the third reaction mixture.

2. The method according to claim 1, wherein the intermediate product is dissolved in step (c) into a low polarity solvent, which has a relative polarity of <0.4 to form the third reaction mixture.

3. The method according to claim 1, wherein the low polarity solvent is selected from ethyl acetate, butyl acetate, tetrahydrofuran, dioxane and toluene.

4. The method according to claim 1, wherein the reaction in the first reaction mixture in step (a) is allowed to proceed at a temperature of 80-95° C..

5. The method according to claim 1, wherein the reactive solvent, which is separated from the first reaction mixture in step (b), is recycled back to step (a).

6. The method according to claim 1, wherein the Cu(I) catalyst precipitated from the second reaction mixture in step (c) is removed prior to step (d).

7. The method according to claim 6, wherein the Cu(I) catalyst precipitated from the second reaction mixture in step (c) is recycled back to step (a).

8. The method according to claim 1, wherein the base is added to the third reaction mixture in step (d) in amount of at least 1 mole equivalent, relative to the amount of the dissolved intermediate product in the third reaction mixture.

9. The method according to claim 1, wherein the base in step (d) comprises an inorganic base.

10. The method according to claim 1, wherein the base in step (d) comprises an organic base.

11. The method according to claim 1, wherein the amount of Cu(I) catalyst in the first reaction mixture is 5-30 mol-% calculated from the amount of the aryl sulphonyl halide in the first reaction mixture.

12. The method according to claim 1, wherein the Cu(I) catalyst is a Cu(I) halide catalyst.

13. The method according to claim 1, wherein the aryl sulphonyl halide is present in the first reaction mixture in step (a), in amount of 0.3-0.7 equivalents relative to the amount of the reactive solvent.

14. The method according to claim 1, wherein the compound according to Formula (I) is recrystallized from an alcoholic solvent after separation step (e).

15. The method according to claim 1, wherein the compound according to Formula (I) is 3-[(4-methylphenyl) sulphonyl]-2-propenenitrile.

16. The method according to claim 4, wherein the reaction in the first reaction mixture in step (a) is allowed to proceed at a temperature of 85-92° C.

17. The method according to claim 5, wherein the reactive solvent, which is separated from the first reaction mixture in step (b), is recycled back to step (a) with a distillation step.

18. The method according to claim 7, wherein the Cu(I) catalyst precipitated from the second reaction mixture in step (c) is recycled back to step (a) without purification.

19. The method according to claim 9, wherein the inorganic base is selected from bicarbonates or carbonates of alkali metals or carbonates of earth alkaline metals, or any mixtures thereof.

20. The method according to claim 10, wherein the organic base is selected from triethylamine, trimethylamine; N-methylmorpholine; N-Page 7 of 8 methylpyrrolidine; N,N-diisopropylethylamine; 1,4-diazabicyclo[2.2.2]octane; 1,8-diazabicyclo[5.4.0]undec-7-ene; or 1,5-diazabicyclo[4.3.0]non-5-ene.
